(19) United States
(12) Patent Application Publication
(10) Pub. No.: US 2007/0015278 A1
(43) Pub. Date: Jan. 18, 2007

(54) METHODS OF ISOLATING DIFFERENTIABLE CELLS FROM PLACENTAL-ASSOCIATED TISSUE AND USES THEREOF

(76) Inventors: Yuling Li, Chapel Hill, NC (US); JoAnne Garvin, Apex, NC (US); Cynthia Pittman, Raleigh, NC (US); Friedrich Hahn, Durham, NC (US); Rulling Xu, Cary, NC (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 11/279,735

(22) Filed: Apr. 13, 2006

Related U.S. Application Data
(60) Provisional application No. 60/670,644, filed on Apr. 13, 2005.

(51) Int. Cl. C12N 5/08

(52) U.S. Cl. 435/366

(57) ABSTRACT

The invention relates to methods of isolating differentiable fetal-derived cells from various tissues of the placenta. Specifically, some of the methods of the present invention relate to isolating differentiable amnion-derived cells, while other methods of the present invention relate to isolating differentiable chorion-derived cells. The present invention also relates to methods of inducing these isolated differentiable amnion-derived and differentiable chorion-derived cells to at least partially differentiate. The present invention also relates to methods of treating insulin deficiencies in a subject by administering these differentiable amnion-derived cells and/or differentiable chorion-derived cells to subjects in need thereof.

This application claims the benefit of U.S. Provisional Application No. 60/670,644, filed Apr. 13, 2005, incorporated herein by reference in its entirety.

### BACKGROUND OF THE INVENTION

1. Field of the Invention

The invention relates to methods of isolating differentiable fetal-derived cells from various tissues of the placenta. Specifically, some of the methods of the present invention relate to isolating differentiable amnion-derived cells, while other methods of the present invention relate to isolating differentiable chorion-derived cells. The present invention also relates to methods of inducing these isolated differentiable amnion-derived and differentiable chorion-derived cells to at least partially differentiate. The present invention also relates to methods of treating insulin deficiencies in a subject by administering these differentiable amnion-derived cells and/or differentiable chorion-derived cells to subjects in need thereof.

2. Background of the Invention

Stem cell therapy has the potential to provide a powerful tool in treating various diseases, particularly degenerative diseases such as Alzheimer's disease, Parkinson's disease and type I diabetes in which pancreatic beta cells are destroyed, causing an increase in blood glucose levels. Due to the current political and cultural climate, however, the use and/or collection of truly “totipotent” stem cells, obtained from the inner cell mass of embryos, is controversial. Alternate sources of stem cells must therefore be sought, if technology is to successfully tap into the enormous potential that stem cell therapy holds.

Alternate sources of potential stem cells or progenitor cells that have recently garnered much attention are the embryonic and fetal organs and extraembryonic membranes, which ultimately derive from the fertilized egg that also gives rise to the embryo and fetus. In particular, the placenta has been studied as a potential source of stem cells that may be useful in therapeutic applications. A normal placenta is composed of a maternal portion and a fetal portion, with each portion being formed by the decidua placentalis and the villi of the chorion, respectively. In addition, the amnion (amniotic membrane) that surrounds the fetus in utero ultimately derives from the fertilized egg.

The amnion is a structure comprised of a single layer of epithelial cells which completely surrounds the fetus as it develops in the uterus. Cells making up the amnion have unique features which make them potentially useful for cell transplantation, including the lack of major histocompatibility complex (MHC) surface antigens on their cell surfaces. The absence of MHC expression on the surface of amnion-derived cells indicates that such cells might be useful in transplantation therapy. One problem with using of placental-derived cells in transplantation, however, is the difficulty associated with their long-term propagation in culture; another problem is a reliable and reproducible mechanism to induce their differentiation into at least partially mature cells capable of providing a therapeutic benefit.

Thus there is a need in the art for a non-controversial source of differentiable stem cells that can be consistently harvested, propagated in culture and induced to differentiate to regenerate specific tissue and/or provide therapeutic benefit to a patient suffering from a degenerative disease.

### SUMMARY OF THE INVENTION

In a first aspect, the invention relates to methods of isolating differentiable amnion-derived cells. These methods comprise subjecting an amniotic membrane to at least one collagenase digestion to produce dissociated amniotic cells, followed by pelleting these dissociated amniotic cells with subsequent washing in a red blood cell lysis buffer. The washed cells are then suspended in a culture media suitable for culturing the differentiable amnion-derived cells. According to one preferred embodiment, this aspect of the invention provides a method of isolating multipotent differentiable amnion-derived cells, the method comprising: subjecting an amniotic membrane to at least one collagenase digestion, wherein the collagenase has a concentration of greater than 1.2 units/ml to about 160 units/ml af activity to produce dissociated amniotic cells; pelleting the dissociated amniotic cells and washing the pelleted cells in a red blood cell lysis buffer; and resuspending the washed cells into a culture media suitable for culturing the multipotent differentiable amnion-derived cells. According to another preferred embodiment, the amniotic membrane is subjected to at least two collagenase digestions. According to another preferred embodiment, the collagenase has a concentration of about 16 units/ml to about 32 units/ml of activity, and more preferably a concentration of about 16 units/ml to about 24 units/ml of activity. According to another preferred embodiment, serum is added to the dissociated amniotic cells, and more preferably, the serum is heat inactivated prior to being added to the dissociated amniotic cells. According to another preferred embodiment, the dissociated amniotic cells are filtered prior to pelleting. According to another preferred embodiment, the red cell lysis buffer is ACK buffer. According to another preferred embodiment, the washed cells are pelleted prior to resuspending the washed cells into a culture media suitable for culturing the multipotent differentiable amnion-derived cells. According to another preferred embodiment, the culture media suitable for culturing the multipotent differentiable amnion-derived cells comprises a media selected from the group consisting of DMEM, F-12, M199 and RPMI.

A second aspect of the present invention relates to methods of isolating differentiable chorion-derived cells. The methods comprise dissociating cells from the chorion of a placenta, and subjecting these dissociated chorionic cells to at least one density gradient centrifugation. The density gradient centrifugation(s) separate(s) differentiable chorion-derived cells from the other cells and debris, and these differentiable chorion-derived can then be collected. According to one preferred embodiment, this aspect of the invention provides a method of isolating multipotent differentiable chorion-derived cells from chorionic tissue, the method comprising: dissociating chorionic cells from the chorionic tissue; subjecting dissociated chorionic cells to at least one density gradient centrifugation; and collecting the multipotent differentiable chorion-derived cells from the density gradient centrifugation. According to another preferred embodiment, the isolated multipotent differentiable chorion-derived cells have a density of between about 1.04 and about 1.10 g/ml, and more preferably have a density of about 1.07 g/ml. According to another preferred embodiment, the isolated multipotent differentiable chorion-derived cells express at least one cell marker selected from the group consisting of: CD90, CD117, SSEA-3, SSEA-4, OCT-4, TRA-1-60 and TRA-1-81, and more preferably at least three cell markers selected from the group consisting of CD90, CD117, SSEA-3, SSEA-4, OCT-4, TRA-1-60 and TRA-1-81 are present in a subpopulation of the isolated multipotent differentiable chorion-derived cells. According to another preferred embodiment, the subpopulation of isolated multipotent differentiable chorion-derived cells does not express OCT-4. According to another preferred embodiment, dissociating chorionic cells comprises mechanically separating the chorionic tissue, or portions thereof, to generate the dissociated chorionic cells; preferably, the amniotic membrane surrounding the chorionic tissue, or portions thereof, has been removed, prior to mechanical separation; and preferably, the chorionic tissue, or portions thereof, has neither been perfused nor exsanguinated prior to mechanical separation. According to another preferred embodiment, dissociating chorionic cells comprises enzymatically separating the chorionic tissue, or portions thereof, to generate the dissociated chorionic cells; preferably, the amniotic membrane surrounding the chorionic tissue, or portions thereof, has been removed, prior to enzymatic separation; and preferably, the chorionic tissue, or portions thereof, has neither been perfused nor exsanguinated prior to enzymatic separation. According to another preferred embodiment, dissociating chorionic cells comprises mechanically and enzymatically separating the chorionic tissue, or portions thereof, to generate the dissociated chorionic cells, wherein the enzyme may be applied before or after mechanical separation. According to another preferred embodiment, dissociated chorionic cells are cultured in a culture media selected from the group consisting of: RPMI and DMEM. According to another preferred embodiment, the at least one density gradient centrifugation comprises at least one selected from the group consisting of iodixanol and sucrose. According to another preferred embodiment, the dissociated chorionic cells are subjected to two or more density gradient centrifugations, which preferably comprise different densities. For example, the different densities may be about 14% iodixanol and about 26% iodixanol.

Yet another aspect of the present invention relates to methods of inducing the isolated differentiable amnion-derived cells or differentiable chorion-derived cells to at least partially differentiate. In one embodiment, the method comprises administering the cells into a subject's pancreas. Preferably, a pharmaceutically acceptable amount of a trophic factor is also administered. Another preferred method involves differentiating such cells, in vitro, into at least partially mature pancreatic beta cells, comprising culturing the cells in culture conditions that promote differentiation. Another preferred method involves differentiating such cells into at least partially mature hepatocytes, comprising administering the cells into a subject's liver, preferably also comprising administering a pharmaceutically acceptable amount of a trophic factor.

Still another aspect of the present invention relates to preparations of differentiable amnion-derived cells and/or differentiable chorion-derived cells isolated from the chorion of a mammalian placenta. According to one embodiment, such cells express at least one cell surface marker, and more preferably at least three cell surface markers, selected from the group consisting of: CD90, CD117, SSEA-3, SSEA-4, OCT-4, TRA-1-60 and TRA-1-81. One embodiment of a preparation of differentiable chorion-derived cells isolated from the chorion of a mammalian placenta does not express OCT-4. The present invention also relates to methods of differentiating these differentiable amnion-derived or chorion-derived cells, in vitro, into more mature cell phenotypes, for example insulin-producing cells. One embodiment involves culturing the cells in placental media.

Still another aspect of the present invention relates to methods for culturing the above-described cell preparations. According to one preferred embodiment, the present invention provides a method for culturing the cells on a cell culture surface comprising at least one compound selected from the group consisting of collagen type I, collagen type III, collagen type IV, collagen type VI, elastin, poly-D-lysine (PDL), poly-D-ornithine (PDO), poly-L-ornithine (PLO) and combinations thereof. In varying embodiments, the surface comprises PLO, PDL and PLO, elastin and PLO, collagen type III and PLO, collagen type 1 and PLO, or elastin and PDL.

Furthermore, the present invention also relates to methods of treating insulin deficiencies in a subject by administering the differentiable amnion-derived cells and/or chorion-derived cells of the present invention to subjects in need thereof, for example by administering such cells into a subject's pancreas.

### BRIEF DESCRIPTION OF THE DRAWINGS

FIG. 1A depicts differences in numbers of cells harvested using various concentrations of collagenase P greater than 1.2 units/ml of activity (0.03%) (“pla63” and “pla64” each refer to a placental preparation number). FIG. 1B depicts effects of increasing collagenase concentration on cell viability.

FIG. 2 depicts effects of collagenase P concentrations of 1.00% to 4.00% (i.e., from 40 units/ml to 160 units/ml of activity) on number of cells harvested.

### DETAILED DESCRIPTION OF THE INVENTION

A normal placenta is composed of a maternal portion and a fetal portion, with each portion being formed by the decidua placentalis and the villi of the chorion, respectively. The present invention relates to methods of isolating differentiable cells from distinct potions of the placenta. As used herein, the term “chorion” or “chorionic tissue” is used to mean at least a portion of the chorionic (or fetal-derived) portion, and not the maternal-derived portion, of the placenta. Whereas the term “placenta” may encompass both the maternal portion and the fetal portion of the placenta. The “amnion” or “amniotic membrane” is the membrane that surrounds the embryo or fetus.

Accordingly, one aspect of the present invention relates to methods of isolating differentiable amnion-derived cells from placental tissue. These methods of isolating differentiable amnion-derived cells comprise subjecting an amniotic membrane to at least one collagenase digestion to produce dissociated amniotic cells, followed by pelleting these dissociated amniotic cells with subsequent washing in a red blood cell lysis buffer. The washed cells are then suspended in a culture media suitable for culturing the differentiable amnion-derived cells.

As used herein, the phrase “amnion-derived cells” is used to mean cells that have been isolated from an amniotic membrane. In one embodiment of the present invention, the amnion, i.e., the amniotic membrane, is removed or separated from all other portions of the embryo or fetus and placenta, prior to performing the methods of the present invention. The amnion is a thin membrane that surrounds the embryo or fetus. When the amnion is initially formed, early during embryonic development, the amnion is in direct contact with the cells of the embryo. As development continues, however, fluid begins to accumulate within the amniotic sac, forcing the membrane apart from the embryo/fetus. Eventually, the fluid forces the amnion to expand such that it adheres to the inner surface of the chorionic portion of the placenta. The differentiable amnion-derived cells of the present invention may be isolated from the amnion at any time after the amnion has formed, including during gestation as well as the termination of pregnancy. Furthermore, the methods of isolating differentiable amnion-derived cells do not require an entire in-tact placenta or even the entire amnion, but only require at least a portion of the amnion. The amniotic membrane from which the differentiable amnion-derived cells are to be isolated can be obtained at the termination of a pregnancy such as post-partum, a miscarriage or an elective abortion.

Another aspect of the present invention relates to methods of isolating differentiable chorion-derived cells. These methods of isolating differentiable chorion-derived cells comprise dissociating cells from the chorion of a placenta and subjecting these dissociated chorionic cells to at least one density gradient centrifugation. The density gradient centrifugation(s) separate(s) differentiable chorion-derived cells from the other cells and debris, and these differentiable chorion-derived cells can then be collected.

As used herein, “chorion-derived cells” is used to mean cells that have been isolated from only the chorionic tissue of a placenta. In addition, the chorion-derived cells of the present invention are not isolated from the amnion surrounding the chorion, nor are the cells isolated from simply exsanguinating the placenta, which may result in isolating cells of maternal origin. In one embodiment of isolating differentiable chorion-derived cells, therefore, the amnion is stripped away from the chorion prior to processing the placenta to isolate the chorion-derived cells. In another embodiment of the methods of isolating differentiable chorion-derived cells, the methods exclude exsanguinating the placenta at any time during the isolation procedures. In addition, the methods of isolating chorion-derived cells, while applicable to an entire placenta, do not require an entire in-tact placenta, comprising both the maternal and fetal portions, but do require at least a portion of the chorion, i.e., the portion of the placenta derived from fetal cells. The placenta or chorion from which the chorion-derived cells are to be isolated can be obtained at the termination of a pregnancy such as post-partum, a miscarriage or an elective abortion.

As used herein, the term “differentiable cell” is used to describe a cell or population of cells that can differentiate into at least partially mature cells, or that can participate in the differentiation of cells, e.g., fuse with other cells, that can differentiate into at least partially mature cells. As used herein, “partially mature cells” are cells that exhibit at least one characteristic of the phenotype, such as morphology or protein expression, of a mature cell from the same organ or tissue. For example, a normal, mature hepatocyte typically expresses such proteins as albumin, fibrinogen, alpha-1-antitrypsin, prothrombin clotting factors, transferrin, and detoxification enzymes such as the cytochrome P-450s, among others. Thus, as defined in the present invention, a “partially mature hepatocyte” may express albumin or another one or more proteins, or begin to take the appearance or function of a normal, mature hepatocyte. Additionally, a “partially mature pancreatic beta cell” may produce or express the proinsulin protein, among others. The ability of the cells to differentiate into at least partially mature cells will not be dependent upon recombinant engineering techniques, such as transfection, though the cells may, of course, be genetically engineered. For the purposes of the present invention, examples of differentiable cells include, but are not limited to, amnion-derived cells and chorion-derived cells.

In one embodiment of the present invention, the differentiable amnion-derived cells and differentiable chorion-derived cells, isolated according to their respective methods, are at least multipotent in their plasticity. In another embodiment, the differentiable amnion-derived cells and differentiable chorion-derived cells of the present invention are at least pluripotent. In still another embodiment of the present invention, the differentiable amnion-derived cells and differentiable chorion-derived cells are at least oligopotent in their plasticity. In yet another embodiment, the differentiable amnion-derived cells and differentiable chorion-derived cells of the present invention are at least unipotent in their plasticity. The “plasticity” of a cell is used herein roughly as it is in the art. Namely, the plasticity of a cell refers to a cell's ability to differentiate into a particular cell type found in tissues or organs from an embryo, fetus or developed organism. The “more plastic” a cell, the more tissues into which the cell may be able to differentiate. “Pluripotent cells” include cells and their progeny, which may be able to differentiate into, or give rise to, pluripotent, multipotent, oligopotent and unipotent cells, and/or several, if not all, of the mature or partially mature cell types found in an embryo, fetus or developed organism. “Multipotent cells” include cells and their progeny, which may be able to differentiate into, or give rise to, multipotent, oligopotent and unipotent progenitor cells, and/or one or more mature or partially mature cell types, except that the mature or partially mature cell types derived from multipotent cells are limited to cells of a particular tissue, organ or organ system. For example, a multipotent hematopoietic progenitor cell and/or its progeny possess the ability to differentiate into or give rise to one or more types of oligopotent cells, such as myeloid progenitor cells and lymphoid progenitor cells, and also give rise to other mature cellular components normally found in the blood. “Oligopotent cells” include cells and their progeny whose ability to differentiate into mature or partially mature cells is more restricted than multipotent cells. Oligopotent cells may, however, still possess the ability to differentiate into oligopotent and unipotent cells, and/or one or more mature or partially mature cell types of a given tissue, organ or organ system. One example of an oligopotent cell is a myeloid progenitor cell, which can ultimately give rise to mature or partially mature erythrocytes, platelets, basophils, eosinophils, neutrophils and monocytes. “Unipotent cells” include cells and their progeny that possess the ability to differentiate or give rise to other unipotent cells and/or one type of mature or partially mature cell type. As used herein, the term “progenitor cell” is used to mean cells and their progeny that can differentiate into at least partially mature cells, but lack the capacity for indefinite self-renewal in culture. Progenitor cells, as used herein, may be pluripotent, multipotent, oligopotent or even unipotent.

The differentiable cells, either amnion-derived or chorion-derived, isolated according to the methods of the present invention are fetal-derived. As used herein, “fetal-derived” indicates that cells are genetically identical to the somatic or germ cells of the fetus, embryo or developed organism, and are not genetically identical to the mother's somatic or germ cells.

As used herein, the term “isolated” or “isolating” or variants thereof, when used in reference to a cell or population of cells means that the cell or population of cells have been separated from a majority of the surrounding molecules and/or materials present which surround the cell or cells when the cell or cells were associated with a biological system (e.g., a functional placenta). The concentration of materials such as water, salts, and buffer are not considered when determining whether a cell has been “isolated.” Thus, the term “isolated” is not intended to imply or indicate a purified population of differentiable amnion-derived cells or chorion-derived cells, nor is it intended to mean a population of cells entirely devoid of debris, non-viable or non-differentiable cells. As used herein, the term “purified,” when used in reference to a cell or population of cells means that the cell or cells have been separated from substantially all materials which normally surround the cell or cells when the cell or cells were associated with a biological system. “Purified” is thus a relative term which is based on a change in conditions in terms of cells and/or materials in close proximity to the isolated cells being purified. Thus, isolated differentiable chorion-derived cells are considered to be purified even if at least some cellular debris, non-differentiable cells, non-viable cells or molecules such as proteins and/or carbohydrates are removed by washing or further processing, after the isolation. The term purified is not used to mean that the all of matter intended to be removed is removed from the cells being purified. Thus, some amount of contaminants may be present along with the purified cells.

As the methods of the present invention provide for the isolation of differentiable cells, the present invention also relates to isolated the differentiable amnion-derived cells and differentiable chorion-derived cells. In one embodiment, therefore, the present invention relates to isolated differentiable amnion-derived cell. In another embodiment, the present invention relates to isolated differentiable chorion-derived cells. The differentiable cells of the present invention must, of course, be viable. As used herein throughout, the term “viable,” when used in relation to an isolated cell or population of cells, is intended to mean a metabolically active cell. The isolated viable differentiable amnion-derived cells or differentiable chorion-derived cells of the present invention may or may not have the ability to divide for a finite or indefinite number of generations; however the cells' ability to divide is not used in determining if the isolated cells are viable for the purposes of the present invention.

The respective methods of isolating differentiable cells of the present invention may initially result in the isolation of one or more subpopulations of viable differentiable cells, based on their phenotype. In other words, the entire population of isolated cells may, at least initially, vary in their phenotype. Thus, in one embodiment of the present invention, individual isolated differentiable amnion-derived or the differentiable chorion-derived cells from the entire population of isolated cells do not exhibit identical phenotypes. In another embodiment of the present invention, however, isolated differentiable amnion-derived cells or the differentiable chorion-derived cells exhibit the identical phenotypes, and are thus considered to be a “pure population” of each of the respective cells. As used herein, a “pure population” of cells is used to mean cells in which a substantial majority, if not all, of the cells of a given population exhibit the identical phenotype. In one embodiment of the present invention the multiple subpopulations of isolated differentiable amnion-derived and differentiable chorion-derived cells are sorted into pure populations, according to their phenotype.

As used herein, the term “phenotype,” in relation to cells is used to mean the collection of proteins that a cell from a particular tissue or organ expresses. For example, the phenotypes of individual isolated cells can be assessed or classified based upon the presence or absence of cell surface markers such as clusters of differentiation (CD factors), which are cell surface antigens. While the phenotype of cells is generally considered to be the collection of proteins that the cell expresses or contains, it may only be necessary to determine the presence or absence of a single protein to adequately classify a cell into a given population or subpopulation. Thus, as used herein, the term “phenotype” is used to connote a particular population or subpopulation to which a cell belongs, based on the presence or absence of at least one protein or portion thereof. For example, the methods of the present invention encompass isolating amnion-derived cells or chorion-derived cells, and determining if the cells are positive or negative for CD90 expression. Continuing the example, the phenotype of a given population or subpopulation of isolated cells may simply be stated as CD90 positive (CD90+) or CD90 negative (CD90−). Of course, the methods of the present invention also contemplate determining the presence or absence of more than one protein to classify a cell into a given population or subpopulation of cells. Examples of CD proteins that may be used to classify cells' phenotypes include, but are not limited to, CD29, CD31 (PECAM), CD34, CD44, CD49, CD54, CD61 (vitronectin receptor), CD71, CD73 (SH3), CD90 (Thy-1), CD105 (SH2), CD133, CD144 and CD166 and CD166. Other proteins may also be used to determine the phenotype of a given cell or population of cells. Examples of other proteins used to classify a cell's phenotype include, but are not limited to, transcription factors such as OCT4, cdx2, and Sox2, transporter proteins such as placental ABC transporter (ABC-p), and other cell surface antigens such as keratin sulphate-associated antigens, TRA-1-60, TRA-1-81 and the stage-specific embryonic antigens (SSEAs), e.g., SSEA-1, SSEA-2, SSEA-3 and SSEA-4. Still more examples of proteins that may be used to classify a cell's phenotype include growth factor receptors such as the receptors for fibroblast growth factor (FGF), transforming growth factor-alpha (TGFa), transforming growth factor-beta (TGFb), activin IIa, and bone morphogenic protein (BMP), as well as the major histocompatibility complex (MHC) proteins, i.e., class I and class II MHC proteins. Still more examples of markers that could be used to identify cells' phenotypes are CK (cytokeratin) 9, CK19, pdx-1, nestin, Pax-6, Nkx2.2, neurofilament, Tau, neuron-specific enolase (NSE), neurofilament protein (NF), microtubule associated protein 2 (MAP2), MAP2 kinase, glial fibrilliary acidic protein (GFAP) and cyclic nucleotide phosphodiesterase. In addition, it may also be possible to detect the presence or absence of portions or domains of proteins, and not the entire protein, to assess or classify a cell's phenotype. For example, some proteins may contain a src-homology domain (SH), such as SH1, SH2, SH3, SH4, etc., the presence or absence of which may be sufficient to adequately assess or classify a cell's phenotype, e.g., SH2+ or SH2−. In one embodiment of the present invention, the isolated differentiable amnion-derived cells are positive for the expression of at least one of CD90, CD117, SSEA-3, SSEA-4, OCT-4, TRA-1-60 or TRA-1-81. In one particular embodiment, at least two markers selected from CD90, CD117, SSEA-3, SSEA-4, OCT-4, TRA-1-60 or TRA-1-81, are present in a subpopulation of isolated differentiable amnion-derived cells. In another particular embodiment, at least three markers selected from CD90, CD117, SSEA-3, SSEA-4, OCT-4, TRA-1-60 or TRA-1-81, are present in a subpopulation of isolated differentiable amnion-derived cells. In one particular embodiment, at least four markers selected from CD90, CD117, SSEA-3, SSEA-4, OCT-4, TRA-1-60 or TRA-1-81, are present in a subpopulation of isolated differentiable amnion-derived cells. As disclosed above, the phenotype of the isolated cells may also be assessed or classified by the absence of particular proteins. For example, in one embodiment of the present invention, at least one population or subpopulation of isolated differentiable amnion-derived cells is negative for the expression of OCT4, i.e., the cells are OCT4−.

Regarding the phenotypes of the differentiable chorion-derived cells, one embodiment of the present invention includes differentiable chorion-derived cells that are positive for the expression of at least one of CD90, CD117, SSEA-3, SSEA-4, OCT-4, TRA-1-60 or TRA-1-81. In another particular embodiment, at least three markers selected from CD90, CD117, SSEA-3, SSEA-4, OCT-4, TRA-1-60 or TRA-1-81, are present in a subpopulation of isolated differentiable chorion-derived cells. In another embodiment, the invention relates to differentiable chorion-derived cells that are negative for the expression of OCT4.

Methods of identifying cells' phenotypes include, but are not limited to, standard immunohistochemistry techniques using antigen-specific antibodies, such as, for example, anti-CD90 antibodies. Other methods of assessing or classifying a cell's phenotype include, but are not limited to, standard blotting techniques such as Western blotting and Northern Blotting, and polymerase chain reaction (PCR) techniques, such as reverse transcriptase-PCR (RT-PCR). Indeed, it should be apparent that indirect methods, such as assays measuring or detecting mRNA, e.g., RT-PCR, can be used to assess or classify a cell's phenotype. Still other methods of assessing or classifying the phenotypes of the isolated chorion-derived cells or isolated amnion-derived cells include flow cytometry techniques, which do not necessarily destroy the cells' viability. Examples of flow cytometry techniques useful for sorting cells based upon their phenotype are disclosed in Practical Flow Cytometry, 3^{rd} Edition, Wiley-liss, Inc. (1995) which is hereby incorporated by reference.

One aspect of the present invention relates to methods of isolating differentiable amnion-derived cells. In particular, the methods of isolating differentiable amnion-derived cells comprise subjecting an amniotic epithelial membrane to at least one collagenase digestion, where the concentration of the collagenase is greater than 1.2 units/ml of activity up to about 160 units/ml of activity. In one embodiment, the concentration of collagenase used in the digestion is from about 1.6 units/ml to about 32 units/ml. In another embodiment, the concentration of the collagenase used in the digestion is from about 16 units/ml to about 24 units/ml. Previous methods disclosed using collagenase concentrations of only up to 1.2 units/ml, most likely because higher concentrations of collagenase are known to affect cell viability. The methods of the present invention, however, overcome cell viability problems associated with using higher concentrations of collagenase. In one embodiment of the present invention, the amniotic membrane is subjected to more than one collagenase digestion, with the concentration of the collagenase being greater that 1.2 units/ml up to about 20 units/ml for at least the first digestion. To perform the digestion, the amnion or a portion thereof is broken apart, or separated, into smaller pieces and placed into a collagenase solution with a concentration of about 0.5% to produce a solution of dissociated amniotic epithelial cells in which individual cells or small clumps of cells from the amnion are suspended in solution. The small clumps may indeed be microscopic. Accordingly, as used herein, the phrase “dissociated amniotic epithelial cells” is used to mean a suspension or suspensions of single cells or small clumps of cells taken directly from the amnion. “Dissociated amniotic epithelial cells” are thus a suspension of cells in a solution that include all cell types and other material found in the normal amnion. It is from this suspension of dissociated amniotic epithelial cells that the differentiable amnion-derived cells are isolated.

In one embodiment of the methods of the isolating differentiable amnion-derived cells, serum is added to the single cell suspension of dissociated amniotic epithelial cells. Serum, such as, but not limited to, bovine serum, newborn calf serum, horse serum and human serum, is a common additive to cell culture solutions and environments, and includes growth factors, albumins and growth inhibitors. In another embodiment of the methods of isolating differentiable amnion-derived cells, the serum that is added to the single cell suspension of the dissociated amniotic epithelial cells is heat-inactivated serum. In yet another embodiment of the methods of isolating differentiable amnion-derived cells, the heat-inactivated serum that is added to the single cell suspension of the dissociated amniotic epithelial cells is heat-inactivated fetal bovine serum. Heat inactivation of serum, e.g., incubating serum at 56° C. for at least about 30 minutes, is a common technique in the art; and heat-inactivated serum is also commercially available.

The methods of isolating amnion-derived cells require pelleting the single cell suspension of dissociated amniotic epithelial cells. As used herein, the phrase “pelleting” is used as it would be in the art. Namely, the single cell suspension must be centrifuged to force the cells into a pellet that is easily removable from any tube containing the cells. Centrifugation of cells is a common technique in the art. The single cell suspension can be centrifuged with enough minimal centrifugal force to coalesce the cells. The cells, however, should not be subjected to such centrifugal forces that the majority of the cells sheer or tear apart, destroying their viability. In one embodiment of the present invention, the single cell suspension is centrifuged at about between 1000 g and 3000 g. In another embodiment, the single cell suspension is centrifuged at about between 1500 g and about 2500 g.

In another embodiment of the methods of isolating differentiable amnion-derived cells, the dissociated amniotic epithelial cells are filtered prior to pelleting, to form a suspension filtrate. The dissociated amniotic epithelial cells can be filtered using any common technique in the art, such as filter papers, sieves and the like. In one embodiment of the present invention, the dissociated amniotic epithelial cells are filtered with a sieve or series of sieves with pores ranging in size from about 55 μm to about 100 μm. In one embodiment, the sieve used for filtering is 75 μm. If the dissociated amniotic epithelial cells are filtered, then the suspension filtrate will be pelleted.

The pelleted cells must then be washed in a buffer capable of lysing any red blood cells that may also be present within the cell pellet. An example of a buffer capable of lysing red blood cells includes, but is not limited to, ammonium chloride potassium (ACK) buffer, which is commercially available. Other red blood cell lysis buffers, such as Erythrlyse™ (Serotec, Inc., Raleigh, N.C., USA) are commercially available. The pH of the red blood cell (RBC) lysing buffer should be between about 6.8 and about 7.6. In particular, the pH should be about between 7.2 and 7.4.

Once the pelleted cells have been washed with RBC lysis buffer, a neutralizing agent is added to the cells, to neutralize the effects of the RBC lysis buffer. An example of a neutralizing agent includes, but is not limited to, phosphate buffer solution (PBS). Once the RBC buffer is neutralized, the cells can then be resuspended in cell culture medium suitable for culturing the isolated differentiable amnion-derived cells. In one embodiment of the methods of isolating differentiable amnion-derived cells, the cells that have been washed in RBC lysis buffer are re-pelleted prior to their being suspended in a suitable cell culture medium. Examples of cell culture media suitable for culturing the isolated amnion-derived cells include, but are not limited to, DMEM, F-12, M199 and RPMI. These cell culture media are commercially available and their preparation is well-known in the art.

The present invention also relates to methods of isolating differentiable chorion-derived cells. The methods isolating differentiable chorion-derived cells comprise dissociating cells from the chorion of a placenta and subjecting these dissociated chorionic cells to at least one density gradient centrifugation. The density gradient centrifugation(s) separate(s) differentiable chorion-derived cells from the other cells and debris, and these differentiable chorion-derived cells can then be collected.

The methods of isolating differentiable chorion-derived cells from the chorion of a placenta comprise dissociating chorionic tissue or cells into a solution of dissociated chorionic cells. As used herein, the phrases “chorionic cells” and “chorionic tissue” are used as they would be in the art; that is, cells or tissue taken or removed only from the chorionic portion of the placenta, and absent the amniontic membrane. The chorionic placenta or a portion(s) thereof is (are) broken apart, or separated, into smaller pieces to prepare a solution in which individual cells or small clumps of cells are suspended in solution. The small clumps may indeed be microscopic. Accordingly, as used herein, the phrase “dissociated chorionic cells” is used to mean a suspension or suspensions of single cells or small clumps of cells taken directly from the chorion. Dissociated chorionic cells are thus a suspension of cells in a solution that include all cell types and other material found in the chorion. It is from this suspension of dissociated chorionic cells that the differentiable chorion-derived cells are isolated. The chorion may be separated mechanically or chemically. Mechanical separation of tissue is readily understood and includes such processes where the tissue is physically separated into smaller and smaller pieces to prepare the cells suspension. Examples of mechanical separation of the chorion include, but are not limited to, cutting, grinding, mixing the tissue to produce the dissociated chorionic cells. Chemical separation of tissue is also readily understood and includes processes where the tissue is placed in solutions, e.g., enzymes solutions, where the solution acts upon the overall integrity of the tissue to produce the cell suspensions. Examples of chemical separation techniques include, but are not limited to, using enzymes such as Liberase™ (Roche Diagnostics, Basel, Switzerland), DNases, RNases and other catalysts, using acids, bases, organic solvents and inorganic solvents. In one embodiment of the methods of isolating differentiable chorion-derived cells, the chorion is mechanically separated to produce the dissociated chorionic cells. In another embodiment of the methods of isolating differentiable chorion-derived cells, the chorion is chemically separated to produce the dissociated chorionic cells. In yet another embodiment of the methods of isolating differentiable chorion-derived cells, the chorion is both mechanically separated and chemically separated to prepare the dissociated chorionic cells.

The solution which contains the dissociated chorionic cells comprises a buffer. Buffer solutions are well known in the art, and examples of buffer solutions in which the dissociated chorionic cells can be suspended include, but are not limited to phosphate buffer solution (PBS), tris-buffered (TBS) and Hanks balanced salt solution (HBSS), and the like. The solution containing the dissociated chorionic cells also comprises a cell culture medium. Cell culture media are well known in the art, and examples of such include, but are not limited to Dulbecco's modified eagle media (DMEM) and RPMI 1640.

The dissociated chorionic cells are then subjected to at least one density gradient centrifugation. As used herein, “at least one density gradient centrifugation” is used mean that the dissociated chorionic cells are subjected to at least one density gradient centrifugation to isolate the differentiable chorion-derived cells. In one embodiment of the methods of isolating differentiable chorion-derived cells, the dissociated chorionic cells are subjected to two or more density gradient centrifugations. If, as the present invention contemplates, the dissociated chorionic cells are subjected to more than one density gradient centrifugation, all density gradient separations may be performed simultaneously by applying one or more additional density gradient solutions in a layered fashion to the dissociated chorionic cells during the same centrifugation separation. For example, two distinct solution gradients, each with different densities, may be layered within a single vessel that also contains the dissociated chorionic cells. Alternatively, if the dissociated chorionic cells are subjected to more than one density gradient centrifugation, additional density gradient separation(s) may be performed separately by applying the additional density gradient solution(s) to the dissociated chorionic cells during independent centrifugation separations.

Methods of density gradient centrifugations are well known in the art, and examples of solutions used in density gradient centrifugations include, but are not limited to, sucrose, silica colloids and iodixanol. In one embodiment of the methods of isolating differentiable chorion-derived cells, the density gradient centrifugation comprises iodixanol and water. Iodixanol is a density gradient that is commercially available as OptiPrep™ (Axis-Shield, Oslo, Norway). Other commercially available density gradient solutions include, but are not limited to PERCOLL™ (polyvinylpyrrolidone-coated silica colloids) (Amersham Biosciences, Piscataway, N.J., USA), RediGrad™ (silane-coated silica colloids) (Amersham Biosciences, Piscataway, N.J., USA) and FICOLL™ (sucrose and epichlorohydrin copolymers) (Amersham Biosciences, Piscataway, N.J., USA).

The density of the solutions used in the density gradients to isolate differentiable chorion-derived cells would be prepared to target the cells with a specific density, and procedures for designing the gradient solutions with specific densities are well-known in the art. In one embodiment of the methods of isolating differentiable chorion-derived cells, the density of the isolated cells is between about 1.04 g/ml and about 1.10 g/ml. In one particular embodiment, the density of the isolated differentiable chorion-derived cells is about 1.07 g/ml.

The present invention also relates to isolated differentiable cells, e.g., differentiable amnion-derived cells or differentiable chorion-derived cells, that have been genetically engineered. The isolated differentiable cells can be genetically engineered to incorporate or introduce a transgene into the differentiable cells. The transgene may incorporated within the genome of the cell (stably transfected) or may not be incorporated into the genome of the cell (transiently transfected). If stably transfected, the transgene may integrate into the genome of the cell of interest, by random integration, or by directed method, e.g., by directed homologous recombination. Such methods of directed homologous recombination are disclosed in U.S. Pat. Nos. 5,272,071, 5,464,764, 5,487,992 and 5,627,059, which are hereby incorporated by reference. More than one transgene may be introduced in to the cells, and these transgenes may be introduced independently or co-introduced or may be located on the same vector.

Examples of polypeptides, proteins or portions thereof coded by the transgene include, but are not limited to trophic factors, previously described herein and other proteins that may provide a therapeutic benefit or a survival or growth advantage to the differentiable cells. For example, a transgene encoding insulin may be introduced into the differentiable amnion-derived cells or the differentiable chorion-derived cells of the present invention.

To introduce the transgene(s) into the differentiable cells of the present invention, a vector, comprising the transgene(s), can be used. The vector may be, for example, a plasmid vector, a single-or double-stranded phage vector, or a single-or double-stranded RNA or DNA viral vector. Such vectors may be introduced into cells by well-known techniques for introducing DNA and RNA into cells. Viral vectors may be replication competent or replication defective. In the latter case, viral propagation generally will occur only in complementing host cells. As used herein, the term “host cell” or “host” is used to mean a differentiable cell of the present invention that is harboring one or more foreign polynucleotides.

Particular vectors for use with the present invention are expression vectors that code for proteins or portions thereof. Generally, such vectors comprise cis-acting control regions effective for expression in a host operatively linked to the polynucleotide to be expressed. Appropriate trans-acting factors are supplied by the host, supplied by a complementing vector or supplied by the vector itself upon introduction into the host.

A great variety of expression vectors can be used to express proteins. Such vectors include chromosomal, episomal and virus-derived vectors, e.g., vectors derived from bacterial plasmids, from bacteriophage, from yeast episomes, from yeast chromosomal elements, from viruses such as adeno-associated virus, lentivirus, baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. All may be used for expression in accordance with this aspect of the present invention. Generally, any vector suitable to maintain, propagate or express polynucleotides or proteins in a host may be used for expression in this regard.

The DNA sequence in the expression vector is operatively linked to appropriate expression control sequence(s) including, for instance, a promoter to direct mRNA transcription. Representatives of such promoters include, but are not limited to, constitutive promoters such as and tissue specific or inducible promoters. Examples of eukaryotic promoters include, but are not limited to the promoter of the mouse metallothionein I gene (Hamer et al., J. Mol. Appl. Gen. 1:273-288 (1982)); the TK promoter of Herpes virus (McKnight, Cell 31:355-365 (1982)); the SV40 early promoter (Benoist, et al., Nature (London) 290:304-310 (1981)); and the vaccinia virus promoter. All of the above listed references are incorporated by reference herein. Additional examples of the promoters that could be used to drive expression of a protein include, but are not limited to, tissue-specific promoters and other endogenous promoters for specific proteins, such as the albumin promoter (hepatocyte), a proinsulin promoter (pancreatic beta cells) and the like. In general, expression constructs will contain sites for transcription, initiation and termination and, in the transcribed region, a ribosome binding site for translation. The coding portion of the mature transcripts expressed by the constructs may include a translation initiating AUG at the beginning and a termination codon (UAA, UGA or UAG) appropriately positioned at the end of the polypeptide to be translated.

In addition, the constructs may contain control regions that regulate, as well as engender expression. Generally, such regions will operate by controlling transcription, such as repressor binding sites and enhancers, among others.

Vectors for propagation and expression generally will include selectable markers. Such markers also may be suitable for amplification or the vectors may contain additional markers for this purpose. In this regard, the expression vectors may contain one or more selectable marker genes to provide a phenotypic trait for selection of transformed host cells. Examples of markers include, but are not limited to, dihydrofolate reductase, hygromycin, mycophenolic acid or neomycin resistance for eukaryotic cell culture. Other methods of selection include but are not limited to selecting for another marker such as thymidine kinase, hypoxanthine-guanine phosphoribosyltransferase and adenine phosphoribosyltransferase.

The vector containing an appropriate nucleotide sequence, as well as an appropriate promoter, and other appropriate control sequences, may be introduced into a differentiable cell of the present invention using a variety of well-known techniques that are suitable for the expression of a desired polypeptide.

Examples of eukaryotic vectors include, but are limited to, pW-LNEO, pSV2CAT, pOG44, pXT1 and pSG available from Stratagene; pSVK3, pBPV, pMSG and pSVL available from Amersham Pharmacia Biotech; and pCMVDsRed2-express, pIRES2-DsRed2, pDsRed2-Mito, pCMV-EGFP available from Clontech. Many other vectors are well-known and commercially available. Selection of appropriate vectors and promoters for expression in a host cell is a well-known procedure, and the requisite techniques for vector construction and introduction into the host, as well as its expression in the host are routine skills in the art.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods in Molecular Biology (1986); Keown et al, 1990, Methods Enzymol. 185: 527-37; Sambrook et al., 2001, Molecular Cloning, A Laboratory Manual, Third Edition, Cold Spring Harbor Laboratory Press, N.Y, which are hereby incorporated by reference.

The present invention also relates to methods differentiating the isolated differentiable cells. In one embodiment of the present invention, isolated differentiable amnion-derived cells are differentiated into at least partially mature cells. In one particular embodiment, the present invention relates to differentiating the differentiable amnion-derived cells into at least partially mature pancreatic beta cells. In another particular embodiment, the present invention relates to differentiating the differentiable amnion-derived cells into at least partially mature hepatocytes. In yet another embodiment of the present invention, the differentiable chorion-derived cells are differentiated into at least partially mature cells. In still another particular embodiment, the present invention relates to differentiating the differentiable chorion-derived cells into at least partially mature pancreatic beta cells. In still another particular embodiment, the present invention relates to differentiating the differentiable chorion-derived cells into at least partially mature hepatocytes.

Methods of differentiating the isolated differentiable cells of present invention include, but are not limited to, (a) culturing the cells in conditions sufficient to induce differentiation into at least partially mature cells, (b) administering the undifferentiated cells in vivo to a subject to induce the differentiable cells to differentiate into at least partially mature cells and (c) combinations thereof.

The methods of inducing differentiation of the differentiable cells include culturing the differentiable cells in cell culture conditions is such a manner as to induce differentiation into at least partially mature cells. The culture conditions include, but are not limited to, culturing the differentiable cells in culture media that is well known in the art, such as DMEM, RPMI, neural growth media etc., that may or may not include trophic factors that may induce differentiation of the cells.

Agents or compounds that elicit cellular responses such as proliferation, differentiation, migration and/or survival the differentiable cells are referred to as “trophic factors.” Such factors include, but are not limited to, cytokines, neurotrophins, growth factors, mitogens, co-factors, and the like, including epidermal growth factor, fibroblast growth factor (acidic and/or basic), platelet-derived growth factor, insulin-like growth factors, ciliary neurotrophic factor and related molecules, glial-derived growth factor and related molecules, schwanoma-derived growth factor, glial growth factor, striatal-derived neuronotrophic factor, platelet-derived growth factor, hepatocyte growth factor, scatter factor (HGF-SF), transforming growth factor-beta and related molecules, neurotransmitters, and hormones. Additional trophic factors that can be employed in the present invention include non-protein compounds or agents, such as vitamins, co-factors, minerals, and organic compounds. Specific factors that can be used to induce differentiation and/or proliferation of the differentiable cells of the present invention include, but are not limited to, Exendin-4, GLP-1, CSF3, IL18, Calcitriol, Dexamethasone, Progesterone, Retinoic acid, Cyclopamine, Folic Acid, ITS, LY294002, Activin A, Activin B, BMPs, TGFB1, Colforsin, Magnesium chloride, Niacinamide, Purmorphamine, Putrescine, Laminin, TNF, n-Butyric acid, Betacellulin, HB-EGF, TGFA, HGF and IGF1. Other trophic factors are known in the art, e.g., Meager & Robinson, Growth Factors: Essential Data (John Wiley and Sons, 1999); McKay & Brown, Growth Factors and Receptors: A Practical Approach (Oxford University Press, 1998); Leroith & Bondy, Growth Factors and Cytokines in Health and Disease, Vol 1A and 1B: A Multi-Volume Treatise (JAI Pr. 1996).

Trophic factors have a broad range of biological activities and their activity and specificity may be achieved by cooperation with other factors. Although trophic factors are generally active at extremely low concentrations, high concentrations of a particular trophic factor, together with high cell density may induce proliferation of some of the differentiable cells of the present invention. For example, growth factors may be useful for enhancing the viability of differentiable cells, as well as for treating disorders by renewal of mature cells from the differentiable cell pool.

The present invention also relates to culturing the differentiable cells in such a manner as to control their state of differentiation. Other aspects of cell culture conditions that may induce or inhibit differentiation of the differentiable cells include, but are not limited to, culturing the differentiable cells on cell culture surfaces to which compounds or agents, such as but not limited to trophic factors, are attached to the cell culture surfaces. Methods of preparing cell culture surfaces that promote attachment, growth and/or survival of various cell type are described in United States Patent Publication Nos. 2004/0062882 and 2005/0058687, and in U.S. Pat. No. 6,129,956, which are all hereby incorporated by reference. In particular, these methods of culturing differentiable cells relate to culturing the differentiable cells in the presence of at least one compound, such as, but not limited to, a trophic factor, a small molecule, a peptide, and components of an extracellular matrix, that is attached to the surface of the co-culture surface. More particularly, at least one compound is attached to the cell culture surface is selected from the group consisting of collagen type I, collagen type III, collagen type IV, collagen type VI, elastin, poly-D-lysine (PDL), poly-D-ornithine (PDO) and combinations thereof. The cell surfaces can also be designed with specific constituents to inhibit fibroblast attachment, growth and/or survival. Thus, in one embodiment of the methods culturing the differentiable cells, the differentiable cells are cultured in such conditions that may inhibit the attachment, growth and/or survival of any non-differentiable cells, e.g., fibroblasts, that may be remaining in the cell preparation, while stimulating the attachment, growth and/or survival of the differentiable cells. For example, the cell culture conditions may include, but are not limited to, culturing the cells on surfaces that are prepared such that the surfaces promote attachment of the differentiable cells to the cell culture surfaces, while inhibiting (or at least not promoting) the attachment, growth and/or proliferation of non-differentiable cells that may be inadvertently present in the culture. In particular, one embodiment of the present invention relates to culturing differentiable cells on culture surfaces comprising PLO, or culture surfaces comprising PDL and PLO. In other particular embodiments, the cell surfaces upon which the differentiable cells are cultured comprise Elastin and PLO, or Elastin and PDL, or collagen type III and PLO or collagen type 1 and PLO.

One embodiment of the methods of culturing the differentiable cells of the present invention comprises culturing the differentiable cells on fabricated cell surfaces in the presence of additional cell types, which may include, but are not limited to, fibroblasts or mesenchymal cells.

The present invention also relates to methods of administering the differentiable amnion-derived cells and/or the differentiable chorion-derived cells to a subject in such a manner as to induce differentiation of the administered cells into at least partially mature cells. As used herein, the term “administer” or variations thereof, in reference to cells, is used to mean that cells are given to or placed within a subject. The administration of the cells to the subject can take place in a variety of forms including, but not limited to, injecting the cells into the subject, surgically implanting the cells and as part of a pharmaceutical composition. Injection sites of the subject include, but are not limited to, subcutaneous, intravenous and intraperitoneal. The site of injection site could be more specific, such as within specific tissue or organ such as the liver, the kidney, the pancreas, a specific portion of the brain or nervous system, bone or bone marrow and the spleen, just to name a few. Similarly, sites of implantation can also include more general areas, or more specific areas such as around or within a specific organ or tissue of the subject. As used herein, the term “subject” is used interchangeably with “patient” and is used to mean an animal, in particular a mammal, and more particularly a human or non-human primate.

In one embodiment of the present invention, isolated differentiable amnion-derived cells are induced to differentiate into at least partially mature beta cells, with the induction method comprising administering the isolated differentiable amnion-derived cells within the pancreas of a subject. As used herein the term “within” as it relates to an administration site is used to mean within, surrounding, beside or in close proximity to a tissue, organ or organ system. In another embodiment of the present invention, isolated differentiable amnion-derived cells are induced to differentiate into at least partially mature hepatocytes, with the induction method comprising administered the isolated differentiable amnion-derived cells within the liver of a subject.

The present invention also provides for methods of inducing differentiation of the differentiable cells in situ, with the induction method comprising administering the differentiable cells, in an undifferentiated state, to a subject and also administering to the subject a pharmaceutically effective amount of an agent that induces differentiation of the administered differentiable cells. Agents that induce differentiation of the administered cells have been described herein and include such agents as the trophic factors. As used herein, the phrase “pharmaceutically effective amount” is to mean an amount of an agent or compound that elicits a cellular response, without excessive side effects.

In another aspect, the methods of differentiating the differentiable cells of the present invention also include the combination of culturing the cells to induce differentiation, followed by administering the cells to a subject. Thus, in one embodiment of the present invention, the differentiable amnion-derived cells are cultured to induce differentiation into at least partially mature cells, prior to their administration into the subject. In another particular embodiment, the differentiable chorion-derived cells are cultured to induce differentiation into at least partially mature cells, prior to their administration into the subject.

The present invention also relates to methods of treating maladies in a subject by administering the differentiable cells to a subject in need of treatment thereof. In one embodiment, the present invention relates to treating a subject for insulin deficiency, with the treatment method comprising administering a therapeutically effective amount of the differentiable amnion-derived cells of the present invention to a subject in need of treatment thereof. In another embodiment, the present invention relates to treating a subject for insulin deficiency, with the treatment method comprising administering a therapeutically effective amount of the differentiable chorion-derived cells of the present invention to a subject in need of treatment thereof. In yet another embodiment, the present invention relates to treating a subject for insulin deficiency, with the treatment method comprising inducing differentiation of the differentiable amnion-derived cells of the present invention to at least partially mature pancreatic beta cells and administering a therapeutically effective amount of the at least partially mature pancreatic beta cells to a subject in need of treatment thereof. In still another embodiment, the present invention relates to treating a subject for insulin deficiency, with the treatment method comprising inducing differentiation of the differentiable chorion-derived cells of the present to at least partially mature pancreatic beta cells and administering a therapeutically effective amount of the at least partially mature pancreatic beta cells to a subject in need of treatment thereof.

When the cells are administered to a patient, one or more immunosuppressive agents may be administered to reduce or prevent rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell co-stimulation pathways, such as agents that interfere with the coupling of T-cells and B-cells via the CTLA4 and B7 pathways, as disclosed in United States Patent Publication No. 2002/0182211. Examples of immunosuppressive agents include, but are not limited to, cyclosporine A, myophenylate mofetil, rapamicin, and anti-thymocyte globulin. In one embodiment of the present invention, the immunosuppressive drug is administered with at least one other therapeutic agent. The immunosuppressive drug can be administered in a formulation which is compatible with the route of administration of the cells and/or the additional therapeutic agent and is administered to a subject at a dosage sufficient to achieve the desired therapeutic effect. In another embodiment of the present invention, the immunosuppressive drug is administered transiently for a sufficient time.

The following examples are illustrative and are not intended to limit the scope of the invention as defined by the appended claims.

### EXAMPLES

### Example 1

### Isolation of Differentiable Amnion-Derived Cells

A fresh human placenta was rinsed with cold sterile saline on a sieve with the amnion membrane facing up. The amniotic membrane skirt was cut on one side to prevent it from folding back onto the chorion. The incision was made at the base of the umbilical cord, and then cut radially. The amniotic membrane was gently lifted away from the chorion with tweezers. The membrane was then placed in 500 ml of chilled PBS supplemented with 100 IU/ml pen/strep, and rinsed with PBS at 4° C.

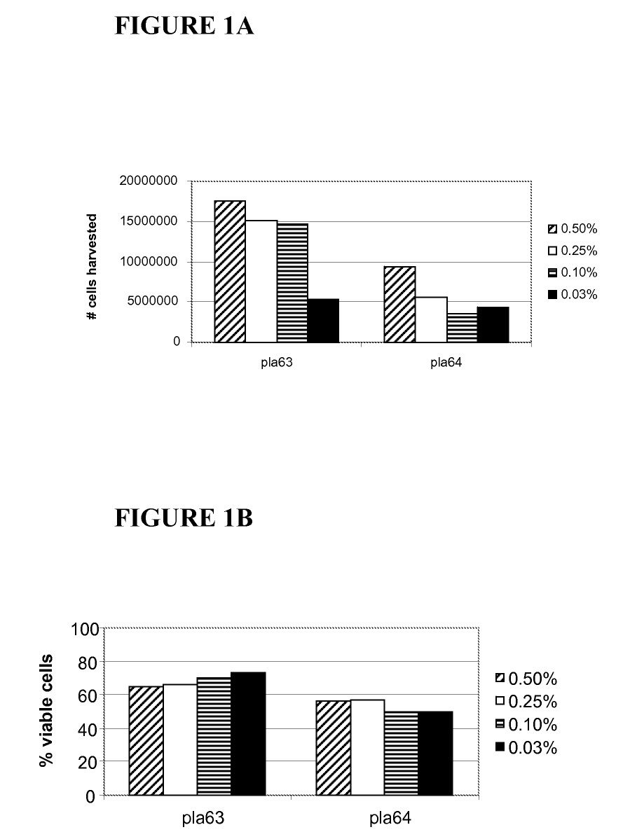

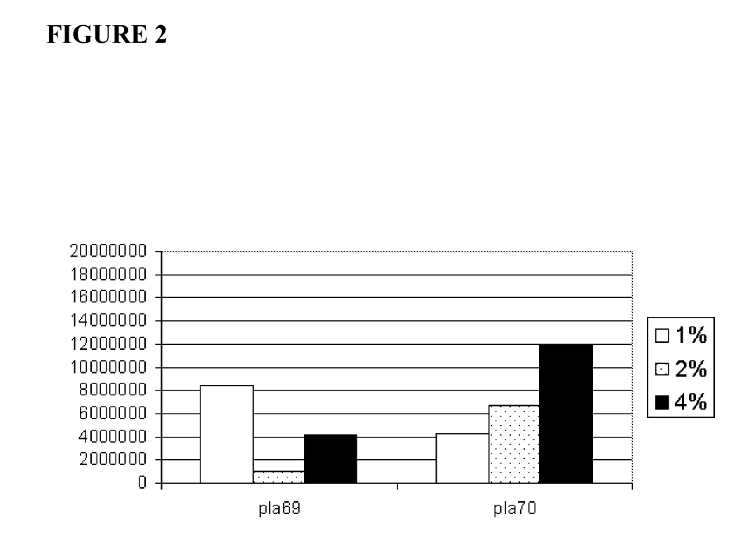

After rinsing, the amniotic membrane was sliced into thin strips while still in buffer. The strips were then incubated in about 10-15 ml of trypsin/EDTA in a TC flask for about 30 minutes at 37° C. in a CO₂ incubator on a rocker. The solution and strips in the TC flask were then transferred into a Petri dish, and 10-15 ml of about 0.04% to about 4.0% collagenase P (1.6 units/ml to about 160 units/ml) in warm PBS was then added to the empty TC flask. FIG. 1A shows the differences in the number of cells harvested using concentrations of collagenase P from 0.03% to about 0.5% (1.2 units/ml to about 20 units/ml); FIG. 1B demonstrates the effects of these collagenase concentrations on cell viability. Furthermore, FIG. 2 shows that collagenase concentrations as high as 1.0%, 2.0% and 4.0% (40 units/ml, 80 units/ml and 160 units/ml, respectively) were also effective in harvesting large numbers of cells (˜4×10⁶-1.2×10⁷) without affecting cell viability. The membrane tissue was then placed back into the TC flask containing the collagenase P and incubated in a CO₂ incubator at 37° C., on a rocker, for about 0.5-2.5 hours to produce a single cell suspension of dissociated amniotic epithelial cells.

After digestion, the single cell suspension was transfer into to 50 ml conical tube and 1 ml of heat-inactivated fetal bovine serum (HI FBS) was added. This tube containing the first digestion was then placed on ice. If the tissue was not completely disaggregated, then 5-10 ml of collagenase P solution was added to the undigested tissue that remained in the TC flask; and the TC flask was re-incubated for about 0.5-5 hours until digestion was complete. Once this second digestion was complete, HI FBS was added to the flask. After this digestion was complete, both the first and second collagenase digests were combined into a 500 ml bottle, and PBS was added to bring the total volume up to 400 ml. The PBS/digest solution was then gently agitated and filtered through 75 μm sieve into a collecting tray. The solution can also be filtered with a sieve with pore sizes of as small as 55 μm with deleterious effects. The contents of the collecting tray, the suspension filtrate, were then placed in a 500 ml centrifuge tube and centrifuged at about 1800-2400 g for about 5-10 minutes at 4° C. The resulting pellet was then removed and washed in about 10 ml of ACK buffer (0.15M NH₄Cl, 10 mM KHCO₃ 0.1 mM Na₂EDTA, at pH of about 7.2 to about 7.4). The ACK suspension was incubated at room temperature and shaken for about 3-10 minutes. Next, PBS was added to neutralize the ACK buffer.

After this washing, the cells were re-pelleted at 1800-2400 g for about 5-10 minutes at about 4° C. If red blood cells were still visible, the cell pellet was again washed with ACK buffer as above and pelleted a third time. Once visibly clear, the pelleted cells were then suspended in a volume of warm Placenta Media (RPMI 1640 w/o glucose, 11.1 mmol/L glucose, 10% FBS, 1% HEPES, 1% NaPyruvate and 71.5 μM beta mercaptethanol 5 ml of pen/strep), and placed in an incubator.

### Example 2

### Transplantation of Differentiable Amnion-Derived Cells into Diabetic Mouse Model

Mice (NOD/SCID) were administered streptozocin (STZ) (200 mg/kg) to induce type I diabetes, as described in Rerup, C. and Tarding, F., Eur. J. Pharmacol. 7(1): 89-96 (July, 1969). Once the blood glucose levels were confirmed at about greater than 300 mg/dl, slow release insulin pellets (0.1 U/d/implant; LinShin Canada Inc., Toronto, Ontario, Canada) were implanted subcutaneously to ensure a euglycemic state for the first few days after the transplantation procedures.

A partial pancreatectomy was then performed on the diabetic mice, and the isolated differentiable amnion-derived cells (about 3×10⁶ cells in 100 μL) of Example 1 were injected into the remaining portion of the pancreas. One week after the cell transplantation procedure, the subcutaneous insulin implants were removed from the mice.

### Example 3

### Glucose Levels and c-Peptide Activity in Post-Transplant Mice

Blood glucose levels can be monitored in transplant mice after cell transplantation, e.g., 4-8 weeks post-transplantation. Blood is extracted from the tail vein or other access point at least once a week and measured. The tail is cleaned with 70% isoproponol and allowed to dry. A small drop of blood exposed by tail vein puncture is placed on, for example, a Freestyle blood glucose strip or other blood glucose biosensor, where glucose concentrations can be read. The tail is then cleaned with a 2% Betadine solution.

In addition, a fasting glucose challenge can be administered at 4, 6 and 8 weeks, post-transplantation and immediately prior to animal sacrifice. Specifically, mice are fasted for 6 hours in a clean cage at the end of the dark cycle, and 100 μl of blood is obtained through the tail vein or other access point to obtain fasting glucose baseline. Next, glucose (10 μl/g bodyweight of a 100 mg/ml stock glucose solution) of is injected intraperitoneally and blood is then collected at 15 and 60 minutes, post-glucose injection. The serum is separated from the remainder of the blood and an ELISA specific for human c-peptide activity is performed to measure its activity.

### Example 4

### Proinsulin Expression in Post-Transplant Mice

Prior to implantation, the cell membranes of the differentiable amnion-derived cells of Example 1 were labeled with Cm-Dil, a red fluor membrane-intercalating dye, (Molecular Probes, Inc, Eugene, Oreg., USA), following the manufacturer's suggested protocol. Briefly, 1×10⁶ cells were suspended per 1 ml of PBS and labeled with 3-5 μM of CM-Dil for 20 minutes at 37° C. Cells were then washed three times with PBS and 3×10⁶ cells were suspended in 100 μl PBS and ready for transplantation. At 6 weeks, post-insulin implant removal, the mice were sacrificed and the pancreases were harvested and frozen in OCT and stored at −80° C. for at least 24 hours. Tissue sections were then cut and mounted for immunohistochemistry analysis. Pancreases can also be harvested at between 4-8 weeks post-insulin implant removal.

Proinsulin protein was detected using mouse anti-human proinsulin monoclonal Ab from Advanced ImmunoChemical (5.2 mg/ml stock, 1/1000 dilution (or 5.2 ug/ml)). The secondary Ab for detection was goat anti-mouse labeled with Alexafluor 488 from Molecular Probes (2 mg/ml stock, diluted 1/400 (5 ug/ml)). Tissue was also stained using 4′,6-diamidino-2-phenylindole (DAPI, Molecular Probes Catalog D3571) to assess gross morphology and highlight cell numbers. To prepare the DAPI staining solution, 2 ml of dimethylsulfoxide (DMSO) was added to 10 mg of stain to prepare a stock solution of 5 mg/ml. The stock solution was then diluted to working solutions of either 0.5 μg/ml or 5 μg/ml. The pancreases in STZ-diabetic mice were positive for human proinsulin protein. Co-staining for human proinsulin and the transplanted cells showed extensive overlap of human proinsulin production and the transplanted cells. The presence of human proinsulin can be attributed to the transplantation of the human differentiable amnion-derived cells that had subsequently undergone differentiation, to at least partially mature pancreatic beta cells, to produce human proinsulin.

### Example 5

### Transplantation of Differentiable Amnion-Derived Cells into Mouse Liver

Prior to implantation, the cell membranes of the differentiable amnion-derived cells of Example 1 are labeled with Cm-Dil, a red fluor membrane-intercalating dye, (Molecular Probes, Inc, Eugene, Oreg., USA), following the manufacturer's suggested protocol, as in Example 4.

NOD/SCID mice are anesthetized and the labeled cells are injected directly into the left lobe of the liver, after midline incision to expose the liver.

At two and six weeks post cell transplantation, the animals are sacrificed and the left lobes of the livers harvested and frozen in OCT, for sectioning and staining for liver specific markers such as, but not limited to, albumin, tyrosine amino transferase, DPP4 and alpha-1 antitrypsin.

### Example 6

### Flow Cytometry Protocol and Results for Characterization of Amnion-Derived Cells

Differentiable cells derived from various portions of the placenta are divided into tubes for immediate analysis or distributed onto plates for extended analysis using flow cytometry. Cells can be stained with single or combinations of antibodies to such antigens as, but not limited to, CD45, CD29, CD90, CD117, CK18, FGF receptor, TGF receptor, BMP receptor, activin receptor, vimentin and alkaline phosphatase.

For immediate analysis of differentiable chorion-derived cells or differentiable amnion-derived cells, the isolated differentiable cells are pelleted in tubes at 1800-2400× g for about 5 to 10 minutes. The cells are then resuspended in flow buffer supplemented with human FcR block (20 ul per 10⁷ cells).

For direct immunofluorescence of cell surface antigens, antibodies are applied at appropriate dilutions and mixed. The Ab-staining solution is applied to the isolated differentiable cells and is then incubated for 30 minutes on ice and protected from light. The isolated differentiable cells are then repelleted as above and rinsed with 5 ml of flow buffer.

For indirect antibody staining of cell surface antigens, the isolated differentiable cells are incubated with primary antibody at the appropriate dilution and duration, then repelleted and rinsed three times with 5 ml of flow buffer. Secondary antibody is then applied, and the cells are then incubated at room temperature for the appropriate dilution and duration and protected from light. The differentiable cells are then pelleted and rinsed three times with flow buffer.

Modifications of the above protocol can be used for antibodies that recognize intracellular or nuclear antigens. Some of these modifications include fixing the cell preparation with 4% paraformaldehyde or other conventional fixation method, and permeablizing the cells with detergents such as 0.05-0.1% Triton X-100. Other commercially available kits offer distinct cell treatments for staining preparation, such as CytoFix/Perm (Becton, Dickinson and Co., Franklin Lakes, N.J., USA).

For time-course analysis, the isolated differentiable cells are plated in wells containing RPMI media with 11.1 mmol/l glucose, 10% ml FBS, 10 mM HEPES, 1 mM sodium pyruvate, 55 μM 2-mercaptoethenol and 50 u/ml penicillin and 50 μg/ml streptomycin) and then incubated at 37° C. for an extended period of time. Cells are then triturated or otherwise released from the surface to achieve a single cell suspension and stained as above, except that volume and centrifugation steps are adapted for use with the plates.

After staining, the cells are pelleted, then brought up to a final volume of 500 μl buffer to (˜10⁷ cells), and flow cytometer is performed on the stained cells for collection of data.

### Example 7

### Isolation of Chorion-Derived Cells from Chorion Using Density Gradient Separation

A human placenta was drained and flushed with PBS P/S (PBS buffer supplemented with pen/strep) and the entire amnion membrane, fat tissue was trimmed away. The placenta was then placed on a large pore sieve and rinsed with copious amounts of cold 1× PBS P/S. Next, the organ was cut into small 1 cubic-inch pieces and placed into a 2L roller bottle, which was then filled with 1L of cold 1× PBS P/S. The roller bottle was placed on a rocker at 4° C., and the PBS P/S was changed about every 30-60 minutes until the PBS P/S was essentially clear. Once the PBS P/S was clear, the PBS P/S was changed one last time and the roller bottle placed back on the rocker at 4° C. for overnight incubation.

After overnight incubation, the placenta pieces were minced in a tissue processor and then transferred to several 500 ml centrifuge tubes. Solutions of Liberase™ and DNase, prepared according to the manufacturers' suggested protocols, were added to the centrifuge tubes; and the tissue was then incubated at 37° C. for 35-40 minutes on a rocker.

After incubation, the tissue/enzyme mixtures were centrifuged at 400 rpm for about 2 minutes to pellet any undigested tissue. The supernatant was removed and transferred into 175 ml tubes. Additional Liberase™ and DNase solutions were added to the undigested tissue and the incubation was repeated. Cold FBS was then added to the first supernatant and mixed by gently inverting the tube. This first supernatant was then stored on ice. The second digestion was then centrifuged as before and the resulting supernatant added to the first supernatant.

The combined supernatants were then centrifuged at 1800-2400 g for about 5 minutes at 4° C., and the fluffy white top layer was removed and discarded. The resulting pellets were then collected pooled into a total of 4 tubes. The resulting supernatants were also pooled and recentrifuged to collect any remaining cells in the supernatants, and these pellets were added to the other cell pellets. The cell pellets were resuspended in 10 ml of ACK buffer, and the mixture was incubated at room temperature for 8 minutes on a rocker. After the 8-minute incubation, PBS was added to bring the total volume up to 400 ml and the solution was spun at 1800-2400 g for about 5 minutes at 4° C. The cells were then resuspended and rewashed in ACK buffer a second time for 3 minutes.

The pelleted cells were then suspended in 50 ml of RPMI P/S without serum or glucose. The cells were then filtered through a series of UV treated sieves, with the pore sizes of the sieves being 1400 μm, 710 μm, 500 μm, 425 μm, 150 μm and 63 μm, and the filtrates were collected in a collection pan. The filtrates were then collected and spun down at 1800-2400 g for about 5 minutes to pellet the cells from the filtrates. The pelleted cells were then suspended in 100 ml of RPMI media. Finally, 100 ml of Optiprep™ was added to 100 ml of the cell suspension to produce a density of about 1.16 g/ml.

50 ml of cold cell suspension/Optiprep™ was poured into centrifuge tubes and set on ice. Next, 50 ml of cold 26% Optiprep™/media (260 ml of Optiprep™ into 340 ml of RPMI media) (density: 1.14 g/ml) was layered on top. Then a second layer of 50 ml of cold 14% Optiprep™/media (140 ml of Optiprep™ into 460 ml of RPMI) (density: 1.07 g/ml) was layered on top of the 26% Optiprep™/media layer. Finally, 25 ml of cold PBS was layered on top of the 14% Optiprep™/media layer, thus creating three layers with at least two density gradients. The multilayered solution was then centrifuged in a swinging bucket rotor at about 810 g for 17 minutes at 4° C., and the tubes were the placed on ice.

The cells in the PBS and 14% Optiprep™/media layers were then harvested from each tube and pooled and spun at 1800-2400 g for 5 minutes at 4° C. The supernatant was then discarded and the cells were resuspended in 5-15 ml of placenta media.

What is claimed is:

1. A method of isolating multipotent differentiable amnion-derived cells, said method comprising:
subjecting an amniotic membrane to at least one collagenase digestion, wherein said collagenase has a concentration of greater than 1.2 units/ml to about 160 units/ml af activity to produce dissociated amniotic cells;
pelleting said dissociated amniotic cells and washing said pelleted cells in a red blood cell lysis buffer; and
resuspending said washed cells into a culture media suitable for culturing said multipotent differentiable amnion-derived cells.

2. The method of claim 1, wherein said amniotic membrane is subjected to at least two collagenase digestions.

3. The method of claim 1, wherein said collagenase has a concentration of about 16 units/ml to about 32 units/ml of activity.

4. The method of claim 1, wherein serum is added to said dissociated amniotic cells.

5. A method of differentiating multipotent differentiable amnion-derived cells into at least partially mature pancreatic beta cells, said method comprising administering multipotent amnion-derived cells isolated according to claim 1 into a subject's pancreas.

6. A method of differentiating, in vitro, multipotent differentiable amnion-derived cells into at least partially mature pancreatic beta cells, said method comprising culturing multipotent differentiable amnion-derived cells isolated according to claim 1 in culture conditions that promote said differentiation.

7. A method of treating insulin deficiency in a subject in need of treatment thereof, said method comprising administering multipotent differentiable amnion-derived cells isolated according to claim 1 into said subject's pancreas.

8. A method of differentiating multipotent differentiable amnion-derived cells into at least partially mature hepatocytes, said method comprising administering multipotent amnion-derived cells isolated according to claim 1 into a subject's liver.

9. A method of isolating multipotent differentiable chorion-derived cells from chorionic tissue, said method comprising:
dissociating chorionic cells from said chorionic tissue;
subjecting dissociated chorionic cells to at least one density gradient centrifugation; and
collecting said multipotent differentiable chorion-derived cells from said density gradient centrifugation.

10. The method of claim 9, wherein said isolated multipotent differentiable chorion-derived cells have a density of between about 1.04 and about 1.10 g/ml.

11. The method of claim 9, wherein said isolated multipotent differentiable chorion-derived cells express at least one cell marker selected from the group consisting of: CD90, CD117, SSEA-3, SSEA-4, OCT-4, TRA-1-60 and TRA-1-81.

12. The method of claim 9 wherein said subpopulation of said isolated multipotent differentiable chorion-derived cells does not express OCT-4.

13. The method of claim 9, wherein said dissociating chorionic cells comprises mechanically separating said chorionic tissue, or portions thereof, to generate said dissociated chorionic cells.

14. The method of claim 9, wherein said dissociating chorionic cells comprises enzymatically separating said chorionic tissue, or portions thereof, to generate said dissociated chorionic cells.

15. The method of claim 9, wherein said at least one density gradient centrifugation comprises iodixanol.

16. The method of claim 9, comprising two or more density gradient centrifugations, wherein said two or more density gradient centrifugations comprise different densities.

17. The method of claim 16, wherein said different densities are about 14% iodixanol and about 26% iodixanol.

18. A cell preparation comprising multipotent differentiable amnion-derived or chorion-derived cells isolated from the chorion of a mammalian placenta.

19. The cell preparation of claim 18, wherein said multipotent differentiable amnion-derived cells express at least one cell surface marker selected from the group consisting of: CD90, CD117, SSEA-3, SSEA-4, OCT-4, TRA-1-60 and TRA-1-81.

20. A method of treating diabetes in a subject in need thereof, said method comprises administering the cell preparation of claim 18 to a subject in need thereof.
